(19) United States
(12) Patent Application Publication
(10) Pub. No.: US 2010/0227798 A1
(43) Pub. Date: Sep. 9, 2010

(54) USE OF THE STAUDINGER LIGATION IN IN VIVO ASSEMBLY OF A BIOLOGICALLY ACTIVE COMPOUND

(75) Inventors: Marc Stefan Robillard, Eindhoven (NL); Holger Cruell, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 12/294,464

(22) PCT Filed: Mar. 20, 2007
(86) PCT No.: PCT/IB07/50969

§ 371 Date: Sep. 25, 2008

(30) Foreign Application Priority Data
Mar. 28, 2006 (EP) 06111847.7

(51) Int. Cl. A61K 38/16; A61K 31/675; A61P 35/00

(52) U.S. Cl. 514/8; 514/81

(57) ABSTRACT

The invention provides methods and tools for the in vivo self-assembly of drugs. This makes it possible to reduce problems associated with the lack of selectivity, reduced solubility and other disadvantages of intact drugs.

### FIELD OF THE INVENTION

The present invention relates to biologically active compounds and methods for delivery of biologically active compounds, which make it possible to circumvent drawbacks or toxic effects of the complete compound by administering it as separate components which allow in vivo re-assembly.

### BACKGROUND OF THE INVENTION

The lack of selectivity of systemically administered drugs is a major problem. Prolonged administration of effective concentrations can be hampered by dose-limiting systemic toxicities. Furthermore, strong side effects in non-target tissue are often observed. Therefore, much effort has been devoted to drug delivery systems that effect drug release selectively at the target site. One concept that has been proposed is to assemble the drug in situ at the target location instead of administering the active drug systemically [Rideout (1986) Science, 233, 561-563; Rideout (1994) Cancer Investigation 12, 189-202]. In this concept, the separate, relatively inactive components of the drug are administered. The active drug is formed through a selective reaction between the two components at sites where both components are taken up. Since the separate components are relatively inactive, toxicity is localized at the target site, thereby minimizing dose-limiting side effects. Furthermore, it has been found that the bimolecular action that is required for a biological effect can lead to a steeper dose-response curve compared to biological effects that arise from the action of a single molecule. This increase of sensitivity to concentration can lead to an increase of the therapeutic window, affording a more effective drug with even less side effects.

To achieve a reaction between the two drug components at or in target cells without side-reactions with other biomolecules, one has to make use of very selective chemical reactions. A requirement for the successful application of such a chemical reaction is that the two participating functional groups must have finely tuned reactivity so that interference with coexisting functionality is avoided. Ideally, the reactive partners would be a biotic, reactive under physiological conditions, and recognize only each other while ignoring their cellular/physiological surroundings (bio-orthogonal). The demands on selectivity imposed by a biological environment preclude the use of most conventional reactions and thus far only the hydrazine-aldehyde Schiff base (hydrazone) formation has been successfully used for in situ drug assembly in vitro. Carbonyl compounds (i.e. ketones and aldehydes) can form reversible Schiff bases with primary amines but the equilibrium in water favors the carbonyl. However, the products from hydrazide or aminooxy groups with carbonyl groups (affording hydrazones and oximes, respectively) are favored in water and are quite stable under physiological conditions. In one report, decanal and octyl aminoguanidine were shown to react in situ to a cell-lysing detergent containing a hydrazone [Rideout (1994) Cancer Investigation 12, 189-202]. Also, using the same reaction, a dicationic protein kinase C inhibitor was assembled in situ from two inactive monocationic phosphonium salts [Rotenberg et al. (1991) Proc. Natl. Acad. Sci. 88, 2490-2494; Rideout et al. (1990) Biopolymers 29, 247-262]. In this case some cancer selectivity was achieved because monocationic tetraphenylphosphonium salts exhibit an increased uptake in cancer cells compared to healthy cells due to higher transmembrane potentials.

Unfortunately the hydrazone or oxime formation in the Schiff reaction does not meet all the above-mentioned requirements for in vivo drug assembly. Most importantly, aldehydes and ketones are not completely abiotic. Biomolecules and metabolites containing these functional groups are present inside cells. As a result, the reaction is not completely bioorthogonal. Also, the optimal pH for these reactions is around 5-6, which is not entirely a physiologically relevant range.

An additional drawback of the hydrazine-aldehyde Schiff system is that the product of the reaction is a hydrazone or oxime. This means that the assembled active drug will always contain such a group. While it is possible to obtain active molecules containing these functionalities, most known drugs or drug-like molecules do not contain these groups. Therefore, the hydrazone/oxmine system is not directly suited to be used universally for the known drugs that could benefit from an in situ assembly approach.

Furthermore, the hydrazine-aldehyde Schiff system does not facilitate active targeting of one or both of the drug components. At best the separate components are designed such that they have an increased (passive) uptake in the target tissue (as shown for the phosphonium drugs Rotenberg et al., 1991, above). While conjugation of one of the components to for example a receptor-targeting peptide is possible, this targeting device will remain attached to the drug after drug assembly. While this does not necessarily preclude the application of this approach in in situ targeted drug assembly, it is not an option for the assembly of unmodified drugs known today.

The Staudinger reaction is an alternative chemical reaction which is biocompatible, abiotic and bio-orthogonal. In the Staudinger reaction (FIG. 1A)—a phosphine and an azide interact to produce an aza-ylide. In the presence of water, this intermediate hydrolyzes spontaneously to yield a primary amine and the corresponding phosphine oxide [Kohn & Breinbauer 2004) Angew. Chem. Int. Ed. 43, 3106-3116]. Bertozzi and co-workers developed a new chemoselective ligation reaction, the Staudinger Ligation (FIG. 1B), based on the classical Staudinger reaction [Saxon& Bertozzi (2000) Science 287, 2007-2010]. A second-generation variant of the ligation reaction (the “traceless” Staudinger ligation) was developed to result in an amide bond from azide and phosphine reagents (FIG. 1C).

The Staudinger ligation has successfully been used for numerous applications, such as peptide ligation, lactam synthesis, bioconjugates, intracellular tagging, metabolic cell engineering and micro arrays. The Staudinger Ligation has been shown to be effective even in vivo (rats) and the azide and phosphine derivatives proved non-toxic in vitro and in vivo [Prescher et al. (2004) Nature, 430, 873-877]. Targeted delivery of prodrugs has been suggested by J C Florent of Institut Curie. According to this concept, a monoclonal antibody bearing one (or more) azide function(s) is administered to the patient. In a second step, a non-toxic prodrug bearing a triphenylphosphine group is administered. It is assumed that when the prodrug would reach the locally bound antibody in the body, a Staudinger reaction would occur between the two functions, with reduction of the azido group into an amine and subsequent liberation of the drug [for further details see http://www.curie.fr/upload/recherche/unites/unit_{—}43/umr176cnrs_ic_{—}2001_gb.pdf, page 2]. However, in view of the instability of the prodrug in cell culture medium, this concept was never realized.

The general usefulness of the Staudinger ligation for the in vivo assembly of drug components has remained unexplored.

### SUMMARY OF THE INVENTION

It is an object of the present invention to provide alternative or improved biologically active compounds, such as drugs or prodrugs, and methods for delivery of biologically active compounds. An advantage of the present invention is that it makes possible to circumvent drawbacks or toxic effects of the intact biologically active compound, while maintaining the desired effect of the intact biologically active compound in situ. This is achieved by administering the biologically active compound as separate components which ensure in vivo (re-) assembly of the biologically active compound.

A first aspect of the invention thus provides a combination of two or more components of an intact biologically active compound, whereby these components have essentially no biological activity, and wherein these components are functionalised with compatible functional groups of the Staudinger ligation, so as to result, upon contact with each other, in the (re-) assembly of the biologically active compound or a biologically active compound having essentially the same activity of the intact biologically active compound.

According to a particular embodiment the combination comprises two components of a biologically active compound.

According to a further particular embodiment of the invention, at least one of the components of the combination of two or more components is targeted, so as to ensure re-assembly of the components at the desired target site. The target components can be inherently targeted. Additionally or alternatively, they can comprise a targeting moiety which ensures targeting of the compound to a desired target site within the body.

According to a further particular embodiment of the invention, at lest one of the components of the combination of two or more components is provided with a detectable label, so as to allow tracking of the component.

A particular embodiment of the invention provides a combination of components of an intact biologically active compound according to the present invention, wherein the biologically active compound has an amide group, and re-assembly of the components results in the generation of this amide group.

The combination of components of a biologically active compound according to the present invention, is intended for separate administration of the components, so as to ensure (re-) assembly of the components in vivo. It is however envisaged that the components of the combination of the invention can be provided as separate pharmaceutical compositions, each in admixture with a pharmaceutically acceptable carrier or excipient, or as one pharmaceutical composition, provided the compounds are physically separated from each other.

The present invention provides combinations of components of biologically active compounds which are functionalized so as to (re-) assemble into an intact biologically active compound or a compound having essentially the same activity as the biologically active compound based on either the traceless and non-traceless Staudinger ligation.

More particularly, the present invention provides combinations of at least two components of an intact biologically active compound, wherein the functional groups provided on the components represent an equal number of functional groups selected from (a) and (b); wherein (a) is a triarylphosphine group which is ortho-substituted with an alkyloxycarbonyl group, whereby the component is linked to the triarylphosphine on one of the remaining positions of one of the aryl groups through a non-reactive linkage or, a triarylphosphine group, whereby the component is linked in such a way that the triarylphosphine is ortho-substituted with the component through a carbonyloxy (—CO—O—) or carbonylthio (—CO—S—) linkage; and wherein (b) is an azide group.

In a further particular embodiment, at least one of the phosphine-bearing components in the combination of the invention is targeted by way of a targeting moiety, which is linked to an aryl group of the triarylphosphine.

According to an alternative embodiment, at least one phosphine-bearing components in the combination of the invention comprises a triarylphosphine group which is ortho-substituted with an alkyloxycarbonyl group, and comprises the targeting moiety linked to the alkyl of the alkyloxycarbonyl group.

The provision of a biologically active compound as a combination of components rather than the intact biologically active compound makes it possible to circumvent certain disadvantages of the intact biologically active compound. Thus, according to one embodiment, the solubility of the components of the combination of the invention is improved compared to the solubility of the corresponding intact biologically active compound.

More particularly, the solubility of the phosphine-bearing component of the combination of the present invention can be increased by modification of the phosphine.

Additionally or alternatively, the stability and/or toxicity of the components of the combination of the invention are improved compared to the solubility of the corresponding intact biologically active compound.

A specific embodiment of the present invention relates to a combination of components of the drug methotrexate, wherein the components correspond to the parts of the methotrexate molecule on each side of the amide group.

Another specific embodiment of the invention relates to a combination of components of the drug bleomycin, more particularly a combination of a first and a second component, wherein the first component corresponds to the metal binding domain of bleomycin and wherein the second component corresponds to the DNA binding domain of bleomycin.

In another aspect, the present invention provides pharmaceutical compositions, comprising a pharmaceutical carrier or excipient in admixture with one component of an intact biologically active compound according to the present invention, which is a component having substantially no biological activity and functionalised with a group which is reactive in a Staudinger ligation.

In yet another aspect, the invention provides a first component of an intact biologically active compound, having substantially no biological activity and further characterized in that it is functionalised with a group which is reactive in a Staudinger ligation, such that contact with a second component results in (re-) assembly of the intact biologically active compound or a compound having essentially the same activity as the intact biologically active compound and whereby the components is further characterized in that it comprises a targeting moiety.

Yet a further aspect of the invention provides a method for preparing components of a biologically active compound according to the present invention. Specific embodiments of this aspect of the invention relate to a method for preparing components of bleomycin, which components are capable of re-assembly into the intact bleomycin upon contact with each other. The methods comprise the following steps: (a) providing the metal binding domain of bleomcyin; (b) providing the DNA binding domain of bleomycin; and (c) providing, on each of these domains of bleomycin, on the position linking these domains in the intact drug, the respective functional groups capable of interacting in a Staudinger ligation;

Yet another aspect of the present invention relates to methods to increase the uptake of an intact biologically active compound, comprising the steps of: (a) providing two components of the intact biologically active compound, which when re-assembled result in the intact biologically active compound or a compound having essentially the same activity as the intact biologically active compound; and (b) providing each of these components with a either a triarylphosphine or an azide functional group capable of reacting together in the Staudinger ligation, to allow the (re-) assembly of the (intact) biologically active compound; Most particularly, the more hydrophobic component of the two components is modified with a triarylphosphine group. Optionally, the triarylphosphine group is further modified by an additional group that increases water solubility of the component.

In yet a further aspect, the present invention provide methods of treatment of diseases which are susceptible to treatment with an intact biologically active compound, which method comprises administering to the patient suffering from such a disease, a combination of the components of the intact biologically active compound, according to the present invention.

The present invention circumvents the above-mentioned limitations of the currently used Schiff reactions and widens the scope of the drug assembly approach by using the selective bioorthogonal Staudinger ligation. The reaction partners of the Staudinger ligation, a phosphine and an organic azide, are completely abiotic, essentially unreactive toward biomolecules inside or on the surfaces of cells, and react in physiological conditions.

Moreover, this reaction is the only ligation reaction that has proven utility in a cellular environment and in rats.

The self-assembly of compounds using the Staudinger ligation, e.g. the in vivo assembly of biologically active compounds or compounds which can be activated into biologically active compounds, has a number of advantages compared to other alleged bioorthogonal reactions: 1) it is a truly bioorthogonal and biocompatible reaction that can also be used inside cells, 2) the traceless ligation allows the traceless assembly of known amide-containing biologically active compounds, 3) the traceless ligation allows traceless targeting of one of the components increasing the effectiveness of the biologically active compound and decreasing the damage to healthy tissue. In other words, the slope of the dose-response curve (and therefore the therapeutic window) can be increased even compared to the two component systems based on the hydrazine-aldehyde Schiff system known today.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference Figures quoted below refer to the attached drawings.

### BRIEF DESCRIPTION OF THE DRAWINGS

FIG. 1 shows a schematic representation of the Staudinger reaction (A), the Staudinger ligation (B) and the traceless Staudinger ligation (C).

FIG. 2 shows a schematic representation of the reaction of two components of a biologically active compound functionalised with a phosphine and an azide according to an embodiment of the present invention.

FIG. 3 shows the non-traceless Staudinger ligation.

FIG. 4 shows, in accordance with a particular embodiment of the invention, the reaction of the functionalised DNA binding domain of bleomycin with the functionalised metal binding domain of bleomycin according to an embodiment of the present invention.

FIG. 5 shows, in accordance with a particular embodiment of the invention, the reaction of the functionalised components of methotrexate according to an embodiment of the present invention.

FIG. 6 The targeting device that can be used in combination with many anticancer drugs is shown in FIG. 6.

FIG. 7 shows, in accordance with a particular embodiment of the invention, the reaction of functionalised components of Piroxicam according to an embodiment of the present invention.

FIG. 8 illustrates the formation of Pteridine phosphine.

### DETAILED DESCRIPTION OF THE PREFERRED EMBODIMENTS

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The terms or definitions used herein are provided solely to aid in the understanding of the invention.

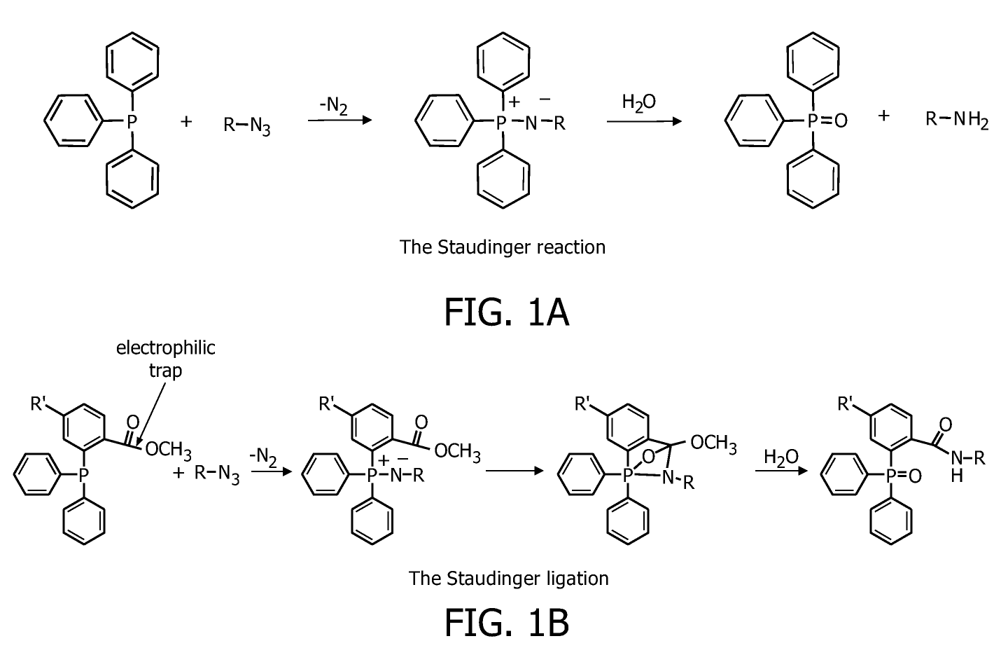

The present invention presents novel applications of the Staudinger ligation, more particularly in the context of drug delivery. The “Staudinger ligation” as referred to in the present application refers to a modification of the Staudinger reaction, resulting in the ligation of two molecules. In the “Staudinger reaction”, a reaction occurs between a phosphine and an azide to produce an aza-ylide (FIG. 1A). In the presence of water, this intermediate hydrolyzes spontaneously to yield a primary amine and the corresponding phosphine oxide. The phosphine and the azide react with each other easily in water at room temperature.

In the Staudinger ligation, the Staudinger reaction has been modified to circumvent the hydrolysis of the aza-ylide intermediate. This can be achieved using in two general approaches. In a first approach, a phosphine bearing an electrophylic trap, e.g. methyl ester, was designed that enables the intramolecular rearrangement of the unstable nucleophilic aza-ylide intermediate into a stable adduct before hydrolysis gets a chance. Thus, in this approach the Staudinger ligation proceeds by reaction of this modified and bioconjugated triarylphosphine with an azide conjugate, after which intramolecular cyclization gives an amide bond and phosphine oxide. This ligation is referred to as the “non-traceless” Staudinger ligation (FIG. 1B), as the ligation product contains an appended triphenylphosphine oxide residue.

A “traceless” Staudinger ligation has been developed to generate a simple amide bond from azide and phosphine reagents (FIG. 1C). This reaction utilizes phosphines bearing a transferable acyl group. Reaction with azides generates, after rearrangement of the intermediate aza-ylide and hydrolysis, the amide linked product and a liberated phosphine oxide.

Use of both the “non-traceless” and the “traceless” Staudinger ligation are envisaged within the context of the present invention.

The term “biologically active compound” as used herein generally refers to a molecule with potential biological activity when administered to a patient. Thus it includes all drugs, pharmaceutical compounds, therapeutic enzymes and proteins, etc. Included within the scope of this term are both compounds which are active per se, as well as compounds which are activated upon contact with specific molecules in the body, e.g. activation by endogenous enzymes.

The term “component of a biologically active compound” or “BAC component” as used in the present invention relates to a part of a biologically active compound, which is critical to its activity, but in itself is not biologically active. A component of a biologically active compound is thus by definition only a fraction of the intact active biologically active compound and does not comprise the complete or intact biologically active compound. (Re-) assembly of the components of a biologically active compound according to the present invention can result in a compound which is identical with the intact biologically active compound or which has essentially the same activity as the intact biologically active compound.

As used herein the term “intact” when referring to a biologically active compound is used to refer to the biologically active compound as originally developed and, where applicable, commercialized.

The terms “functionalisation” and “functionalised” as used herein refer to the addition or presence of a functional group, which in the context of the present invention refer to the functional groups which interact in the non-traceless or traceless Staudinger ligation. While specific embodiments are described herein, generally the components of the biologically active compound bearing a phosphine or azide functional group will be referred to as the ‘phosphine-bearing component’ and the ‘azide-bearing component’, respectively. As the ligations always require at least one pair of components, these will generally also be referred to as the ‘first’ and the ‘second’ component, respectively, within such a pair, notwithstanding that more than one phosphine-bearing component and more than one azide-bearing component may be envisaged.

The terms “reduced pharmaceutical activity” or “substantially biologically inactive” as used herein to refer to a component of a biologically active compound, indicate an activity or therapeutic effect which, as determined via standard assays, is reduced or negligible compared with the activity or therapeutic effect of the intact biologically active compound. The reduced activity of a BAC component can be an activity which is less than 5%, less than 1%, less than 0.1% or up to less than 0.01% of the activity of the intact biologically active compound.

The term “essentially the same activity as the intact biologically active compound”, when referring to the compound obtained in vivo after (re-) assembly of the components of the invention, refers to the fact that the pharmacological or biological activity of said biologically active compound after (re-) assembly is at least 70%, more particularly at least 80%, even more particularly at least 90%, most particularly between 95-100% compared to the intact biologically active compound.

The term “pharmaceutically acceptable carrier or excipient” as used herein in relation to pharmaceutical compositions means any material or substance with which the components of the present invention, may be formulated in order to facilitate their application or dissemination to the locus to be treated, for instance by dissolving, dispersing or diffusing the said component and/or to facilitate its storage, transport or handling without impairing their ability to re-assemble into a biologically active compound. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, pellets or powders. Suitable pharmaceutical carriers for use in the said pharmaceutical compositions and their formulation are well known to those skilled in the art.

In one aspect, the present invention is based on the concept that administration of a biologically active compound in the form of substantially inactive components thereof can have a number of advantages, such as reduced side effects, improved uptake, reduced systemic toxicity etc. Moreover, the present invention provides that by use of the Staudinger ligation to recombine the BAC components in vivo, efficient re-assembly of the components in vivo can be ensured. Finally, the present invention provides that by use of the traceless Staudinger ligation, a traceless targeting can also be ensured.

In general, the nature of the biologically active compound envisaged within the context of the present application is not critical. Thus, biologically active compounds suitable for use in the context of the present invention include but are not limited to antiproliferative/antitumor agents, antibiotics, anti-inflammatory agents, anti-viral agents, antihypertensive agents, and chemosensitizing agents. Nevertheless, it is envisaged that based on the structure and structure-activity relationship, some biologically active compounds may be more easily amenable to the present invention than others. This is described more in detail below.

According to a first aspect of the present invention, substantially inactive components of a biologically active compound are provided which are functionalised, so as to be able to (re-) assemble into the intact biologically active compound or a compound having essentially the same activity as the intact biologically active compound.

The substantially inactive components of a biologically active compound are obtained either by direct synthesis of the components or by breaking up of an intact biologically active compound into components. The selection of the nature of the components is primarily determined by the requirement that all components of the biologically active compound are substantially inactive and/or that the components are such that they do not present (or can be modified to avoid) the inherent disadvantages of the intact biologically active compound. In addition thereto, the selection of the nature of the components will be influenced by different parameters including, but not limited to, the amenability to functionalization according to the invention, size and stability of the components, solubility and the ability to append further moieties, such as targeting moieties (see below) etc. According to a particular embodiment the present invention provides the use of two components of a biologically active compound, whereby upon (re-) assembly of the two components the intact biologically active compound or a compound having essentially the same activity as the intact biologically active compound is obtained. Alternatively, the use of more than two components can be envisaged (e.g. 3 or 4), as long as their appropriate (re-) assembly into the intact biologically active compound or a compound having essentially the same activity as the intact biologically active compound can be ensured. In order to achieve this, the present invention provides that the components are provided with (an even number of) functional groups capable of interacting in the Staudinger ligation, at the (those) position(s) linking the respective components in the intact biologically active compound.

As detailed below, one embodiment of the present invention provides components of a biologically active compound which compound, in its intact form, comprises an amide bond. Components of this compound can be generated, such that, upon (re-) assembly, the amide bond resulting from the ligation corresponds to the amide bond in the intact biologically active compound. The components are then modified to comprise e.g. a triarylphospine group and an azide group, respectively, to allow a traceless ligation between the components.

According to one embodiment of the present invention the components of a biologically active compound are substantially inactive, even prior to their functionalization. In another embodiment, the components of the biologically active compound become substantially inactive as a result of their functionalization.

The present invention provides for assembly of components of a biologically active compound based either on the non-traceless or traceless Staudinger ligation. Depending on the type of ligation envisaged, the nature of the phosphine group which is used to functionalize the first component interacting with the azide group of the second component will vary. More specifically, for the non-traceless Staudinger ligation, the first component is provided with a triarylphosphine group, which is ortho-substituted with an alkyloxycarbonyl group (see also FIG. 1B). The first component of the biologically active compound is linked to the triarylphosphine on one of the remaining positions of one of the aryl groups through a non-reactive linkage. For the traceless ligation, the first component is provided with a triarylphosphine group in such a way resulting a triarylphosphine which is ortho-substituted with the first component of the biologically active molecule through a carbonyloxy (—CO—O—) or carbonylthio (—CO—S—) linkage (see also FIG. 1C). More particularly, in both embodiments, the triarylphosphine is a triphenylphosphine group, which is optionally substituted. Accordingly, depending on the ligation envisaged, the intact biologically active compound will comprise, after (re-) assembly based on these ligations, either an amide bond conjugated to triarylphosphine oxide (non-traceless Staudinger ligation) or an amide bond (traceless Staudinger ligation).

The choice of ligation which is relied upon, will be determined in part by the nature of the intact biologically active compound. For instance, for those pharmacological compounds which are known to comprise an amide bond, or where it is known that the presence of an amide bond does not substantially affect its activity (i.e. results in a compound having essentially the same activity as the intact biologically active compound), the traceless Staudinger ligation will be preferentially relied upon. Accordingly, a particular embodiment of the invention relates to the provision of substantially inactive components of a biologically active compound that naturally contains an amide bond, whereby the components are functionalised with a phosphine group bearing a transferable acyl group and an azide, respectively. According to this embodiment re-assembly of the components of the biologically active compound will result in a pharmacological compound or an essentially intact pharmacological compound which is identical or nearly identical to the intact pharmacological compound. A non-exhaustive list of pharmaceutical compounds with amide bonds comprises bio-active (poly)peptides, e.g. peptide hormones and enzymes, receptor binders, and drugs derived from amino-acid like compounds, oligonucleotides, carbohydrates, but also certain classes of inorganic compounds such as condensation polymers, e.g. the microbicide SAMMA. The use of functionalised components of bleomycin and methotrexate in accordance with the present invention is explained in the examples section.

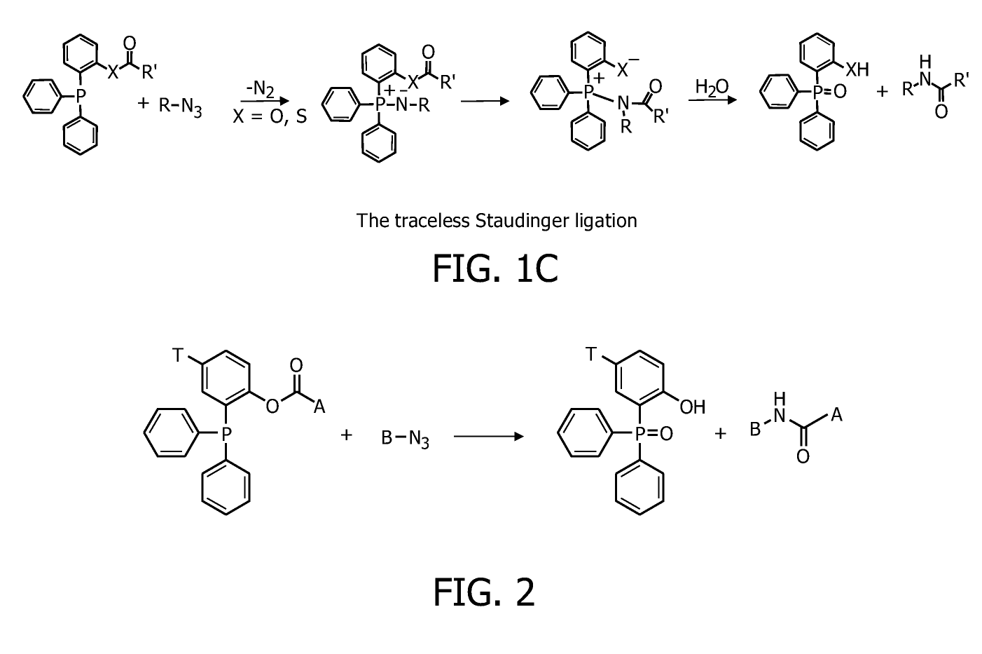

An embodiment of the general concept of the use of the traceless Staudinger ligation in assembly of biologically active compounds according to the invention is illustrated in FIG. 2. An active biologically active compound such as a drug containing an amide bond is divided in two components (A and B) corresponding to the residues on both sides of the amide functionality of the original drug. Component A is functionalised with the traceless Staudinger phosphine probe and Component B is functionalised with an azide. When both components encounter each other in vivo the resulting traceless ligation between these two components affords the intact amide-containing drug.

According to another embodiment the invention provides substantially inactive components of a biologically active compound which either naturally contains an amide bond conjugated to a phosphine oxide, or a biologically active compound for which the presence of an amide bond conjugated to a phosphine oxide is known (or has been determined) not to significantly affect the biological or therapeutic activity of the biologically active compound, e.g. resulting in an a biologically active compound having essentially the same biological activity as the intact biologically active compound. According to this embodiment the invention provides substantially inactive components of a biologically active compound whereby the first component is functionalised with a triarylphosphine either through an ester or thioester linkage in ortho position on the aryl relative to the phosphor (traceless), or through a non-reactive linkage on an aryl of the triarylphosphine which is ortho substituted with an alkyloxycarbonyl or alkylthiocarbonyl (non-traceless) and the second component is functionalised with an azide.

According to a particular embodiment, one or more of the functionalised components of the biologically active compound of the present invention can be a targeted component. More specifically, the component can be inherently targeted based on its selective uptake or distribution in one or more specific organs, optionally in combination with specific routes of administration.

Alternatively, functionalised components of the biologically active compound which do not per se have a specific distribution or affinity can be targeted by the attachment of a targeting moiety. Different targeting moieties are envisaged within the context of the present invention to ensure the targeting of the one or more components of the biologically active compound to a tissue or organ of interest. Examples include, but are not limited to antibodies or antibody fragments (such as, but not limited to Fab₂, Fab, scFV), or other compounds which are capable of specifically binding to or are specifically taken up by specific cells, tissues or organs, such as, but not limited to octreotide and derivatives, VIP, MSH, LHRH, chemotactic peptides, bombesin, elastin, peptide mimetics, carbohydrates, monosaccharides, polysaccharides, viruses, drugs, chemotherapeutic agents, receptor ligands, agonists and antagonists, cytokines, hormones, steroids. Examples of organic compounds suitable as targeting moieties envisaged within the context of the present invention are, or are derived from, estrogens, e.g. estradiol, androgens, progestins, corticosteroids, paclitaxel, etoposide, doxorubricin, methotrexate, folic acid, and cholesterol.

According to a particular embodiment of the present invention, the targeting moiety is capable of specifically binding to a receptor such as the ligand of the receptor or a part thereof which still binds to the receptor, e.g. a receptor binding peptide in the case of receptor binding protein ligands.

Other examples of targeting moieties of protein nature include interferons, e.g. alpha, beta, and gamma interferon, interleukins, and protein growth factor, such as tumor growth factor, e.g. alpha, beta tumor growth factor, platelet-derived growth factor (PDGF), uPAR targeting protein, apolipoprotein, LDL, annexin V, endostatin, and angiostatin.

Alternative examples of targeting moieties include DNA, RNA, PNA and LNA which, by nature of their sequence or part thereof are capable of specifically hybridizing to a target nucleotide.

According to a particular embodiment of the invention, small lipophilic primary moieties are used which can bind to an intracellular target.

According to the present invention, the targeting moiety is attached to one or more of the components of the biologically active compound so as to ensure targeted delivery thereof upon administration to the body. Targeting of only one of the components is sufficient, whereby either the azide-comprising drug component or on the phosphine-comprising component can be targeted. In certain embodiments a targeting moiety is provided on both the azide- and the phosphine-comprising component of the biologically active compound.

According to a first embodiment, the targeting moiety is attached through a functional group on the component of the biologically active compound itself. Using this approach the presence of the targeting moiety on the component is not affected by the re-assembly of the biologically active compound, and the targeting moiety thus remains attached to the biologically active compound after re-assembly. Notwithstanding, the targeting moiety can be partially or completely removed by other factors, such as the metabolism of the cell.

According to another embodiment, the targeting moiety is provided on the triarylphosphine group of the phosphine-comprising component of the biologically active compound in the traceless Staudinger ligation ('T′ in FIG. 2). More particularly, the targeting moiety can be linked to one of the phenyl groups of triphenylphosphine. Using this approach, the targeting moiety will be released from the re-assembled biologically active compound when the Staudinger ligation takes place between the two components.

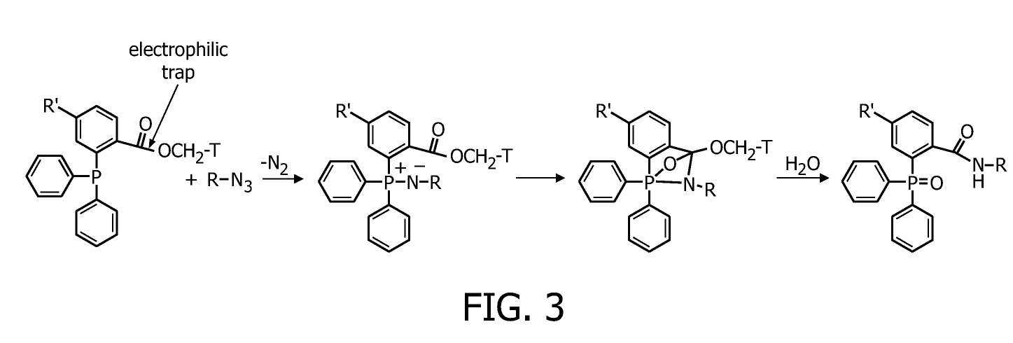

In the non-traceless Staudinger ligation, the targeting moiety can alternatively be provided on the alkyl group of the alkyloxycarbonyl group involved in the Staudinger ligation. In this embodiment, the targeting moiety is also no longer present in the biologically active compound upon re-assembly of the components (FIG. 3).

According to a further aspect of the invention functionalised components of a biologically active compound are each provided individually and separately, each optionally in admixture with a pharmaceutical acceptable carrier or recipient. The functionalised components can be provided as a kit of parts for combined, sequential or consecutive administration to the human or animal body. Typically the components of the present invention will be provided as a combination of two or more components of one biologically active compound. Different types of packaging or formulation are envisaged, provided said packaging or formulation prevents contact of the individual components with each other before administration.

Administration of the different components of one biologically active compound can be via the same or different routes. The different routes of administration for the components of the biologically active compound envisaged within the context of the present invention include, but are not limited to oral administration, topical administration (mucous membrane, skin), injection (such as intravenous, subcutaneous, intramuscular, intraperitoneal, intramammary), respiratory (inhalation, intranasal, intratracheal), or rectal administration.

Accordingly, a further aspect of the invention relates to a method of treatment, which comprises administering to a subject two or more components of a biologically active compound wherein the components of the biologically active compound are functionalised to ensure re-assembly of the biologically active compound based on a Staudinger ligation.

Different administration regimes are envisaged to optimalize the efficiency of the re-assembly of the biologically active compound after the administration of each of the components. Most particularly, if one of the functionalised components is targeted (either inherently or by way of a targeting moiety) to the desired organ or tissue, this moiety can be administered prior to the administration of the functionalized component which is not or less targeted to that desired organ or tissue.

Thus, according to one embodiment, the method of administration generally comprises two steps:

a first step wherein the functionalised component of the biologically active compound having the greatest target specificity is administered to the body.

a second step wherein the further functionalised component(s) of the biologically active compound is(are) administered.

Optionally, a time period is envisaged between step 1 and step 2 to allow the first component of the biologically active compound to reach its target and/or to allow clearance of the remaining component.

According to the present invention, the re-assembly of the biologically active compound will occur only at those places where both complementary functionalised components meet each other.

Different variants and modifications of the above-described components and process of administration can be envisaged. For example, one or both (or more) components of the biologically active compound can be further modified with a detectable label to allow tracking of the distribution of one or both (or more) components. This makes it possible to adjust the moment of administration of the second functionalised component based on the outcome of the tracking of the first component, or to adjust the amount of the second component to be administered.

Particular examples of detectable labels of the imaging probe are contrast agents used in traditional imaging systems such as MRI-imagable agents, spin labels, optical labels, ultrasound-responsive agents, X-ray-responsive agents, radionuclides, (bio)luminescent and FRET-type dyes. Exemplary detectable labels envisaged within the context of the present invention include, and are not necessarily limited to, fluorescent molecules, e.g. autofluorescent molecules, molecules that fluoresce upon contact with a reagent, etc., radioactive labels; biotin, e.g., to be detected through reaction of biotin and avidin; fluorescent tags, imaging agents for MRI comprising paramagnetic metal, imaging reagents, e.g., those described in U.S. Pat. Nos. 4,741,900 and 5,326,856) and the like.

The MRI-imagable agent can be a paramagnetic ion or a superparamagnetic particle.

The ultrasound responsive agent can comprise a microbubble, the shell of which consisting of a phospho lipid, and/or (biodegradable) polymer, and/or proteins like human serum albumin. The microbubble can be filled with fluorinated gasses or liquids.

The X-ray-responsive agents include but are not limited to Iodine, Barium, Barium sulfate, Gastrografin or can comprise a vesicle, liposome or polymer capsule filled with Iodine compounds and/or barium sulfate.

Moreover, detectable labels envisaged within the context of the present invention also include peptides or polypeptides that can be detected by antibody binding, e.g., by binding of a detectable labeled antibody or by detection of bound antibody through a sandwich-type assay.

In one embodiment the detectable labels are small size organic PET and SPECT labels, such as ¹⁸F, ¹¹C or ¹²³I.

The general two step method described above may require high amounts of the non-targeted second functionalised component to be administered, in order to ensure that the two components encounter each other in the body and that a sufficient amount of the active re-assembled biologically active compound is formed. To address this potential inconvenience, specific administration methods are envisaged.

In one embodiment, both complementary functionalised components are embedded in a degradable matrix with a targeting moiety. This matrix physically separates the components from each other. After targeting and subsequent degradation of the matrix the functionalised components are in each other's proximity and can react in an efficient way.

In another embodiment, both functionalised components are present as solid particles in a targeted vesicle. After targeting and disruption or disintegration of the vesicle, both components can react with each other.

In yet another embodiment, both functionalised components are present in solution in a vesicle under conditions wherein the Staudinger ligation cannot occur (e.g. masking of one or both of the functional groups). After disruption or disintegration, the compounds are released and, further to liberation of the functional group(s), can react. Alternatively, an embodiment is envisaged whereby the vesicle is permeable, and the conditions within the vesicle change gradually and the biologically active compound is formed within this microenvironment. When the vesicle is disrupted or disintegrated, the re-assembled biologically active compound is.

In yet another embodiment, the complementary functionalised components of the biologically active compound (one or both targeted if necessary) are connected by a degradable linker (e.g. peptide, lipid or oligosaccharide). After targeting and degradation of the linker by enzymes of the body, both components can react and form the biologically active compound.

The present invention makes it possible to ensure administration of a biologically active compound which, when administered in its intact form, presents certain disadvantages. More particularly it is envisaged that the administration as inactive components is a significant advantage for biologically active compound which as intact compounds are generally toxic or have side-effects due to activity on non-target tissue. The in vivo assembly of biologically active compounds according to the present invention affords superior disease-selective action of biologically active compounds with diminished side effects. This technology makes it possible to increase the therapeutic window of an array of known drugs, ensuring an increased success rate of therapies based on these drugs, and therefore an increased application/prescription. Thus the present invention provides for components of a biologically active compound of which the toxicity to non-target cells is decreased compared to the toxicity of the intact biologically active compound. More particularly, the toxicity is decreased by 20%, even more particularly by 30%, most particularly by at least 50% as determined in in vitro toxicity studies. Additionally or alternatively, the present invention makes it possible to overcome physical and/or chemical disadvantages of the biologically active compound. For instance, effective distribution of a biologically active compound within the body is often hampered by the limited solubility of the compound in water. Existing methods for drug optimalization to increase water solubility often decrease cellular uptake or membrane diffusion as the hydrophilic/lipophilic balance or the efficacy is changed. Poorly water-soluble biologically active compounds are therefore administered using drug delivery systems such as liposomes or emulsions that offer a hydrophobic compartment for high drug uptake. These delivery vehicles can carry a high payload of hydrophobic biologically active compounds and provide an extended blood half-life. A disadvantage of liposomal or emulsional drug delivery is their high liver uptake, which leads to an increase in liver toxicity. Using the concept of the present invention it is possible to increase the water solubility of biologically active compounds. The solubility of the one or more functionalised components of the biologically active compound of the present invention can either be inherently higher or can be modified by addition of functional groups. Thus the present invention provides for components of a biologically active compound of which the solubility is improved compared to the solubility of the intact biologically active compound. More particularly, the solubility is improved by at least 20%, even more particularly by at least 30%, most particularly by at least 50% or more. According to a particular embodiment, the most hydrophobic component is functionalised as the first component with a phosphine group on which functional groups are provided which affect solubility of the component, e.g., (poly)alcoholic groups and carbohydrates (also referred to herein as a “water-soluble tag”). As upon reaction with the second component in the Staudinger ligation, the phosphine group is removed, the additional groups will not influence the activity of the reassembled biologically active compound.

### EXAMPLES

### Example 1

### In Vivo Assembly of Bleomycin

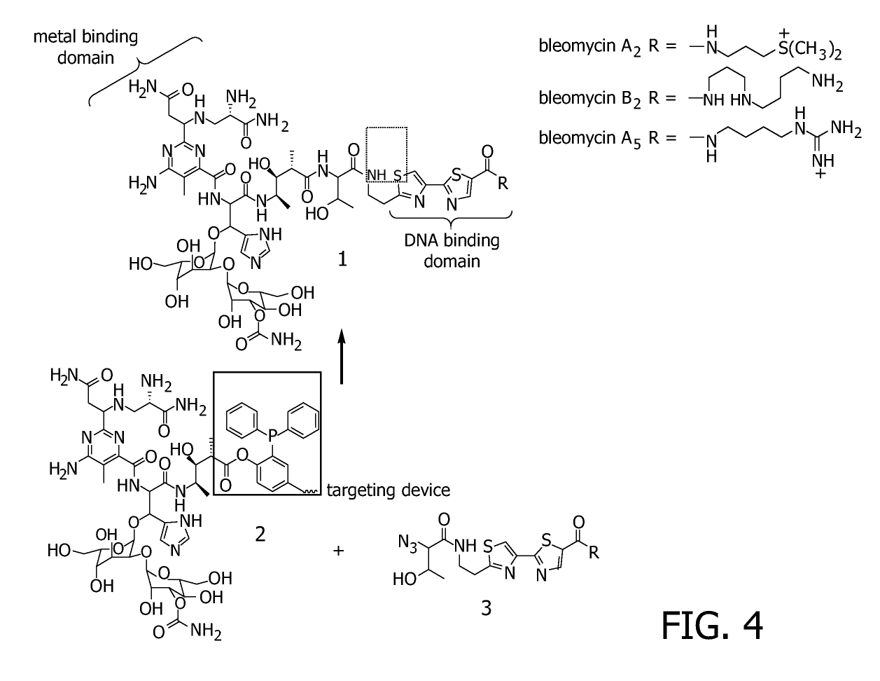

A mixture of bleomycin, a family of antitumor antibiotics, is used for cancer chemotherapy (predominantly A₂ and B₂, FIG. 4). When combined with a metal ion cofactor bleomycin can cleave DNA. The drug comprises various essential domains, two of which are the metal binding domain and the DNA binding domain. In this example bleomycin 1 is provided as two separate components containing the metal binding domain or the DNA binding domain, respectively, resulting in two DNA-inactive components (2 and 3). DNA cleaving compound 2 is functionalised with a traceless Staudinger phosphine probe (boxed, full line) which is conjugated to a cancer-selective targeting device. DNA binding compound 3 is functionalised with an azide (boxed, dotted line). After systemic administration of 2 and its accumulation at the cancer site, component 3 is injected. Selective reaction between 2 and 3 at the target site will result in the simultaneous release and activation of the assembled drug.

### Example 2

### In Vitro and In Vivo Assembly of Methotrexate

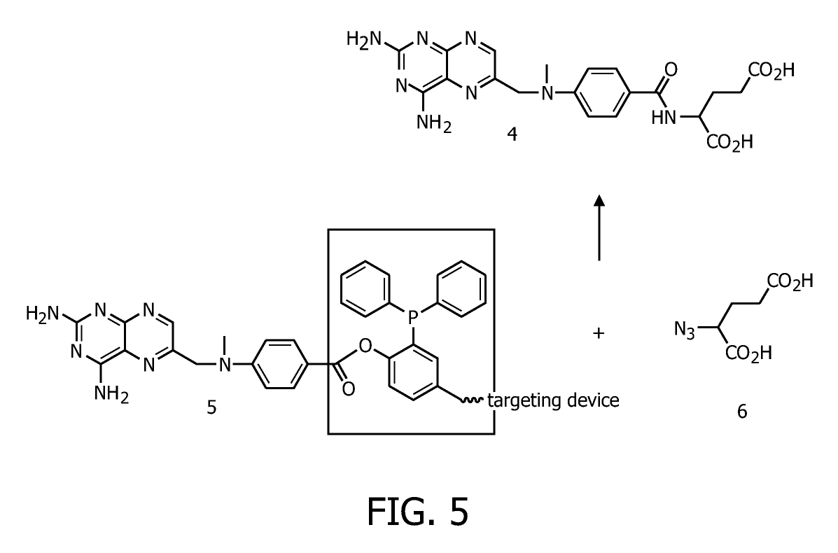

Methotrexate (compound 4, FIG. 5) is divided into two components (compound 5, 6), each corresponding to one part of the intact molecule on a side of the amide bond. These components are functionalised with the traceless Staudinger ligation reaction partners in such a way that reaction between these two components results in the re-assembly of methotrexate.

Evaluation of the reassembly of the methotrexate compound in vitro is assessed based on the reduction of proliferation in a human sarcoma cell line as follows:

Human sarcoma cells (HT-1080) are seeded in 96-multiwell flatbottom microtiter plates. The plates are incubated at 37° C., 5% CO₂ for 24-48 h prior to drug testing to allow cell adhesion. Stock solutions of the two methotrexate components 5 and 6 are freshly prepared and the dilutions are prepared in complete medium. The range of the concentrations used is 0.1 nM-10 uM. Each concentration is tested in quadruplicate using 50 μl/well of component 5 and 50 μl/well of component 6, added to the 100 μl of complete medium containing the cells. In the control groups only 100 μl of complete medium is added or 50 μl of complete medium mixed with 50 μl of either component 5 or 6. The plates are incubated for 72 hrs and the evaluation of cell proliferation is performed by the MTT colorimetric assay.

Methotrexate (compound 4, FIG. 5) is used as an anticancer chemotherapeutic as well as in the treatment of arthritis and other rheumatic conditions. It works by blocking the enzyme dihydrofolate reductase, thereby interfering with the production of a form of folic acid that is important for actively growing cells. Analogous to example 1, the therapeutic window of Methotrexate therapy is increased by applying a targeted in situ assembly approach. To this end, the methotrexate compound is divided into two components as described above. The triarylphosphine-bearing component is further functionalized with a targeting moiety, ensuring targeting of this component to cancer cells. Ligation of the triarylphosphine-containing component (5) with the azide-comprising component (6) occurs at the targeted cells, whereby the targeting device is released.

### Example 3

### In Vivo Assembly of Biologically Active Compounds Using Metabolites as Targeting Moiety

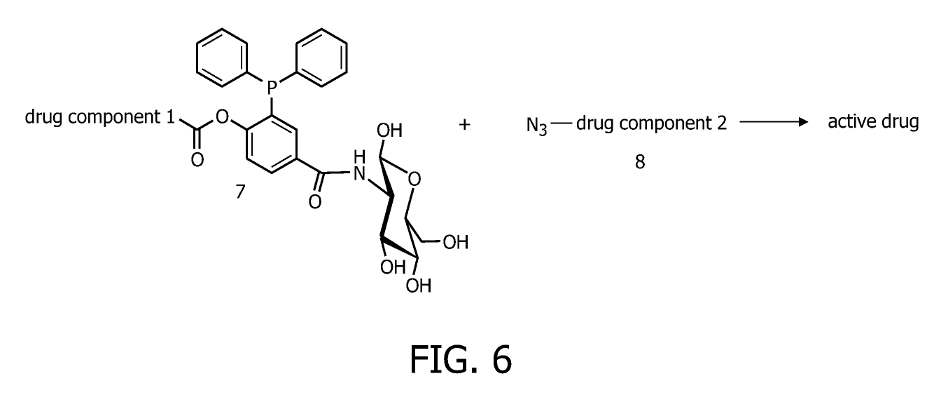

Many anti-cancer drugs can be targeted using metabolites of the glucose pathway. The cellular glucose metabolism pathway can recognize the phosphine-glucose conjugate 7 (FIG. 6). After cellular uptake these modified saccharides are trapped and accumulate inside the cell as result of a first metabolic phosphorylation step. After systemic administration, glucose is optimally accumulation in tissue with a high glucose uptake (e.g. tumor tissue) and will be optimally cleared from other non-target tissues and blood. The second drug component (8′, FIG. 6) is then administered. Component 8 is conjugated to component 7, which is trapped in the cells via the traceless Staudinger ligation. Upon ligation, the active amide-containing drug is re-assembled, while the phosphine-glucose group is removed from the molecule.

As an extension of this approach, the azide-containing drug component (8) can also be functionalised with a glucose moiety. In this manner both inactive drug components are specifically targeted to the tumor tissue. However, the reassembled drug in this embodiment is a glucose-drug conjugate, which remains trapped inside the cell.

### Example 4

### Use of the Staudinger Ligation for Improving the Water Solubility of Biologically Active Compounds

According to the present invention, poorly water-soluble biologically active compounds are provided as two components of which the water solubility is higher or modified to be higher than the intact biologically active compound. This is of particular interest for biologically active compounds that carry an amide function.

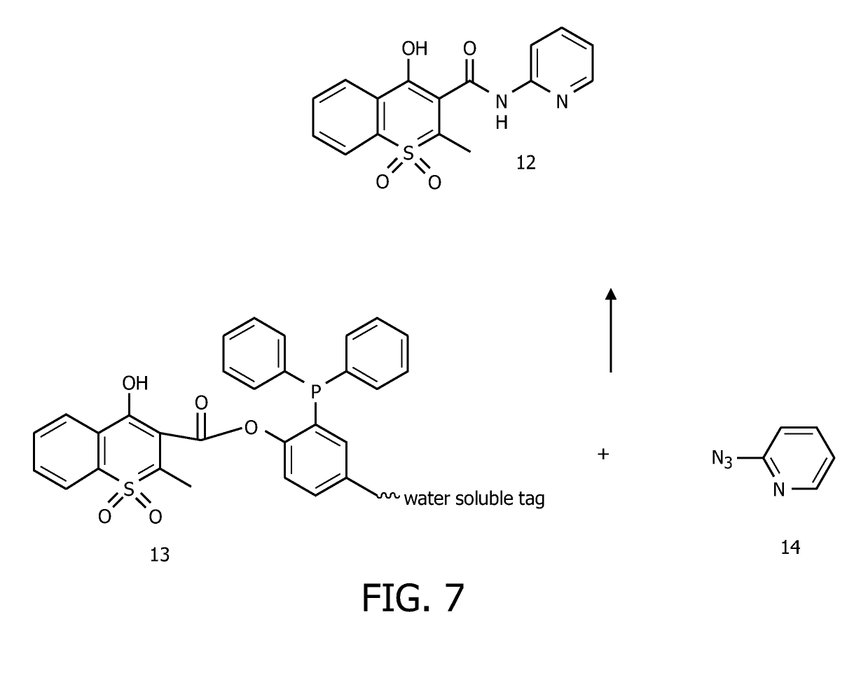

Piroxicam (compound 12, FIG. 7), is a compound comprising an amide function, which is provided as two components (components 13 and 14 in FIG. 7) that are modified with an azide and a triphenylphosphine derivative, respectively. The latter is attached to the more hydrophobic part of the drug and carries an additional side group to render the whole construct water-soluble. Optionally, the properties of the azide-carrying component are also adjusted to obtain increased water solubility compared to the intact drug.

Upon treatment of the patient with pain, the two components of Piroxicam are co-administered. The biologically active compound is re-assembled in vivo via a Staudinger ligation.

### Example 5

### Pteridine Phosphine

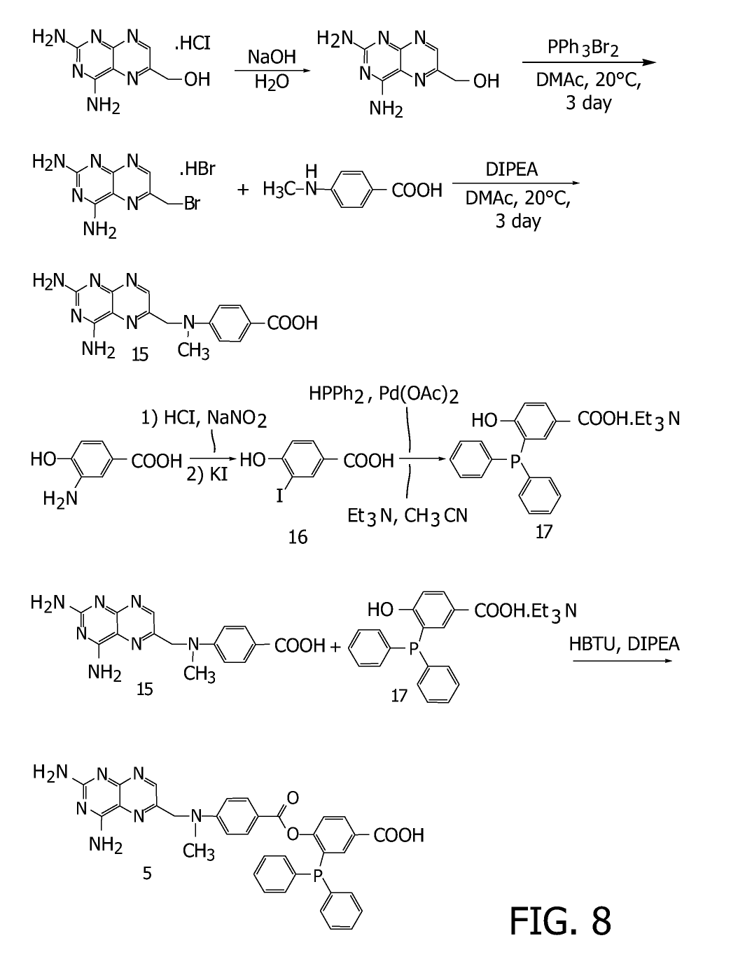

This example is illustrated in FIG. 8.

### Azido-L-glutamic acid (6),

Sodium azide (8.83 g; 136 mmol) was dissolved in water (25 mL), dichloromethane (37.5 mL) was added and the mixture was vigorously stirred at 0° C. Triflic anhydride (7.89 g; 27.9 mmol) was added over a few minutes, and the reaction mixture was stirred for 30 min at 0° C., and 1 hr at 20° C. Then, the organic layer was isolated, and the aqueous layer was extracted with dichloromethane (2 times 15 mL). The combined organic layers containing triflyl azide were washed with sat. Na₂CO₃ (10 mL).

L-Glutamic acid (2.06 g; 14.0 mmol), K₂CO₃ (5.80 g; 42.0 mmol), and Cu(II)SO₄.5H₂O (35 mg; 0.14 mmol) were dissolved in water (50 mL), and methanol (90 mL) and the triflyl azide solution were added. The reaction mixture was stirred for 20 hr at 20° C. The volatile solvents were evaporated in vacuo, water (250 mL) was added, the solution was neutralized with 6 M hydrochloric acid (ca. 80 mL), and phosphate buffer of pH=6.2 (250 mL) was added. The mixture was washed with ethyl acetate (4 times 200 mL), and the pH was adjusted to 2 by addition of 6 M hydrochloric acid (ca. 6 mL). This was extracted with ethyl acetate (3 times 200 mL), and the combined organic layers were dried with MgSO₄, filtered, and evaporated to dryness to yield the product as a colorless liquid (1.98 g; 82%).

¹H-NMR (CDCl₃): δ 4.13 (t, 1H, J=6.5 Hz), 2.4-2.7 (br. m, 2H), 2.22 (m, 2H) ppm. ¹³C-NMR (CDCl₃): δ 178.8, 175.8, 60.5, 29.6, 26.0 ppm. FT-IR (ATR): ν 2927, 2107, 1704, 1413, 1208, 1056, 913, 857, 792 cm⁻¹.

### 4-[N-[(2,4-diamino-6-pteridinyl)methyl]-N-methylamino]benzoic acid (15)

2,4-diamino-6-(hydroxymethyl)pteridine hydrochloride (4.40 g; 19.19 mmol) was dissolved in hot water (150 mL) and the solution was neutralized by addition of 1 M NaOH (ca. 20 mL). The precipitate was filtered, washed with water, and dried in vacuo over P₂O₅. Subsequently, this was suspended in DMAc (25 mL), and triphenylphosphine dibromide (18.12 g; 42.92 mmol) was added. The turbid reaction mixture was stirred for 20 hr at 20° C. 4-(Methylamino)benzoic acid (2.95 g; 19.50 mmol) and DIPEA (5.04 g; 39.00 mmol) were added, and the turbid reaction mixture was stirred for 3 days at 20° C. Then, it was poured in 0.33 M NaOH-solution (250 mL), the precipitate was filtered off, and the filtrate was neutralized by addition of 10% acetic acid in water (ca. 20 mL). The precipitate was filtered, washed with water, triturated with methanol (30 mL), and dried in vacuo over P₂O₅, to yield the product as a beige/orange powder (3.48 g; 56%).

¹H-NMR (DMSO): δ 8.59 (s, 1H), 7.53 (d, 2H, 8.8 Hz), 7.7 (br. s, 1H), 7.5 (br. s, 1H), 6.83 (d, 2H, 8.8 Hz), 4.79 (s, 2H), 3.23 (s, 3H) ppm. FT-IR (ATR): ν 3335, 3194, 2964, 1651, 1597, 1292, 1187 cm⁻¹.

### 3-Iodo-4-hydroxybenzoic acid (16)

3-Amino-4-hydroxybenzoic acid (10.00 g; 65.30 mmol) was dissolved in conc. hydrochloric acid (100 mL, and a solution of sodium nitrite (4.73 g; 68.56 mmol) in water (30 mL) was added dropwise at 0° C. The reaction mixture was stirred at 0° C. for 30 min, and subsequently, a solution of potassium iodide (54.20 g; 326.5 mmol) in water (100 mL) was added dropwise at 0° C. After stirring at 0° C. for 30 min, the reaction mixture was heated to 70° C. until no more gas evolved (ca. 1 hr). The mixture was extracted with ether (3 times 250 mL), and the combined organic layers were washed with 1 M NaHSO₃ (100 mL). The aqueous layer was back-extracted with ether (150 mL). The combined organic layers were dried over Na₂SO₄, filtered, and evaporated to dryness. The remaining solid was recrystallized from a mixture of methanol (30 mL) and water (70 mL) to yield the product as brown crystals (8.95 g; 52%). ¹H-NMR (MeOD): δ 8.34 (d, 1H, J=2.2 Hz), 7.85 (dd, 1H, J=2.2, 8.6 Hz), 6.86 (d, 1H, J=8.6 Hz) ppm. FT-IR (ATR): ν 3271, 1679, 1574, 1362, 1288, 1239, 1195, 767 cm⁻¹.

### Triethylammonium 3-diphenylphosphine-4-hydroxybenzoate (17)

3-Iodo-4-hydroxybenzoic acid (4.00 g; 15.15 mmol) was suspended in acetonitrile (90 mL), and triethylamine (9.19 g; 90.9 mmol), palladium acetate (0.17 g; 0.76 mmol), and diphenylphosphine (4.23 g; 22.73 mmol) were added. The reaction mixture was heated to 90° C. under an argon atmosphere for 20 hr. The precipitate was filtered, washed with acetonitrile and dried, to yield the product as a beige solid (5.06 g; 79%).

¹H-NMR (DMSO): δ 7.78 (dd, 1H, J=2.2, 8.6 Hz), 7.61 (m, 1H), 7.38 (br. m, 6H), 7.20 (br. m, 4H), 6.84 (dd, 1H, J=4, 8.6 Hz), 2.60 (q, 6H, J=7.0 Hz), 1.00 (t, 9H, J=7.0 Hz) ppm. ³¹P (DMSO): 6-17.0 ppm. FT-IR (ATR): ν 2409, 1825, 1584, 1529, 1390, 1354, 1339, 1286, 1123, 791, 743, 700 cm⁻¹.

### Pteridine phosphine (5),

4-[N-[(2,4-diamino-6-pteridinyl)methyl]-N-methylamino]benzoic acid (15) (0.650 g; 2.00 mmol) was suspended in DMAc (15 mL). DIPEA (0.79 g; 6.14 mmol) and HBTU (0.92 g; 2.43 mmol) were added, and the reaction mixture was stirred at 20° C. for 20 hr. The precipitate was filtered, washed with DMAc and ether, and dried to yield the intermediate HOBt-ester (0.668 g; 1.51 mmol).

Triethylammonium 3-diphenylphosphine-4-hydroxybenzoate (17) (0.639 g; 1.51 mmol) was suspended in DMSO (5 mL), and potassium tert-butoxide (0.339 g; 3.02 mmol) was added. To this solution was added a solution of the intermediate HOBt-ester in DMSO (5 mL), and the reaction mixture was stirred at 20° C. for 20 hr. Subsequently, it was filtered, poured in water (100 mL), neutralized with acetic acid and centrifuged. The mother liquor was decanted, and the precipitate was stirred in water (100 mL), and again centrifuged and decanted. The residue was dissolved in acetone (100 mL) and recrystallized at −20° C., to yield the product as a yellow powder (0.360 g; 29%). ¹H-NMR (DMSO): δ 8.60 (s, 1H), 8.01 (dd, 2H, J=2, 8.6 Hz), 7.65 (br. s, 2H), 7.50 (d, 2H, J=9.2 Hz), 7.42 (br. m, 8H), 7.23 (m, 3H), 6.76 (d, 2H, J=9.2 Hz), 6.62 (br. s, 2H), 4.81 (s, 2H), 3.25 (s, 3H) ppm. ³¹P-NMR (DMSO): −16.6 (product), +23.4 (small, phosphine oxide) ppm. FT-IR (ATR): ν 3319, 3182, 3053, 1717, 1634, 1600, 1523, 1435, 1367, 1272, 1244, 1172, 1115, 1063, 1045, 742, 691 cm⁻¹. LC-MS (CH₃CN/H₂O): tᵣ=8.9 min: m/z=630.1 (M+H)⁺; tᵣ=7.5 min: m/z=646.0 (phosphine oxide).

1. A combination of two or more components of an intact biologically active compound, said components having essentially no biological activity, wherein said components are functionalised with compatible functional groups of the Staudinger ligation, so as to result, upon contact with each other, in the re-assembly of a biologically active compound having essentially the same activity of the intact biologically active compound.

2. The combination of claim 1, characterized in that at least one of said components is targeted.

3. The combination of claim 2, wherein said targeted component(s) comprise a targeting moiety.

4. The combination according to claim 1, characterized in that at least one of said components comprises a detectable label.

5. The combination of claim 1, wherein said intact biologically active compound has an amide group, and re-assembly of said components results in the generation of said amide group.

6. The combination of claim 1, wherein said components are provided as separate pharmaceutical compositions, each in admixture with a pharmaceutically acceptable carrier or excipient.

7. The combination according to claim 1 wherein said functional groups are reactive in the traceless Staudinger ligation.

8. The combination according to claim 1, wherein said functional groups represent an equal number of functional groups selected from (a) and (b) wherein:
a) a triarylphosphine group which is ortho-substituted with an alkyloxycarbonyl group, whereby said component is linked to said triarylphosphine on one of the remaining positions of one of the aryl groups through a non-reactive linkage or, a triarylphosphine group, in such a way that said triarylphosphine is ortho-substituted with the component through a carbonyloxy (—CO—O—) or carbonylthio (—CO—S—) linkage and
b) an azide group.

9. The combination according to claim 1, comprising 2 components.

10. The combination according to claim 8, wherein at least one of said phosphine-bearing components is targeted by way of a targeting moiety.

11. The combination according to claim 10, wherein said targeting moiety is linked to an aryl group of said triarylphosphine.

12. The combination according to claim 10, wherein said at least one phosphine-bearing component comprises a triarylphosphine group which is ortho-substituted with an alkyloxycarbonyl group and wherein said targeting moiety is linked to said alkyl of said alkyloxycarbonyl group.

13. The combination according to claim 1, wherein the solubility of said components is improved compared to the solubility of said intact biologically active compound.

14. The combination according to claim 13, wherein at least one of said component(s) is a phosphine-bearing component and wherein said phosphine of said phosphine-bearing component is modified to increase the solubility of said phosphine-bearing component.

15. The combination according to claim 1, wherein the stability and/or toxicity of said components are improved compared to the stability of the intact biologically active compound.

16. The combination according to claim 1, comprising two components of methotrexate, wherein said components correspond to the parts of the methotrexate molecule on each side of the amide group.

17. The combination according to claim 1, which comprises two components of bleomycin, wherein the first of said components corresponds to the metal binding domain of bleomycin and wherein the second of said components consists of the DNA binding domain of bleomycin.

18. A pharmaceutical composition comprising a pharmaceutical carrier or excipient in admixture with a component of an intact biologically active compound, wherein said component has substantially no biological activity and wherein said component is functionalised with a group which is reactive in a Staudinger ligation.

19. A first component of an intact biologically active compound, having substantially no biological activity characterized in that said component is functionalised with a group which is reactive in a Staudinger ligation, such that contact with a second component results in re-assembly of a biologically active compound having essentially the same activity as said intact biologically active compound and further characterized in that it comprises a targeting moiety.

20. A method for preparing components of bleomycin capable of re-assembly into the intact bleomycin upon contact with each other, said method comprising
a) providing the metal binding domain of bleomcyin,
b) providing the DNA binding domain of bleomycin,
c) providing, on each of said domains of bleomycin, on the position linking these domains in the intact drug, the respective functional groups capable of interacting in a Staudinger ligation,

21. A method to increase the uptake of an intact biologically active compound, comprising the steps of:
a) providing two components of said biologically active compound, which when re-assembled result in said biologically active compound or a compound having essentially the same activity as said intact biologically active compound,
b) providing each of said components with a either a triarylphosphine or an azide functional group capable of reacting together in the Staudinger ligation, to allow said (re-) assembly; whereby the more hydrophobic component of said two components is modified with a triarylphosphine group.

22. The method according to claim 21 wherein said triarylphosphine group is further modified by an additional group that increases water solubility of said component.
